(19) United States
(12) Patent Application Publication
(10) Pub. No.: US 2023/0190740 A1
(43) Pub. Date: Jun. 22, 2023

(54) AN NK-1 RECEPTOR ANTAGONIST FOR TREATING A DISEASE SELECTING FROM SEPSIS, SEPTIC SHOCK, ACUTE RESPIRATORY DISTRESS SYNDROME (ARDS) OR MULTIPLE ORGAN DYSFUNCTION SYNDROME (MODS)

(71) Applicant: NeRRe Therapeutics Limited, Stevenage (GB)

(72) Inventor: Mike Trower, Stevenage (GB)

(73) Assignee: NeRRe Therapeutics Limited, Stevenage (GB)

(21) Appl. No.: 17/907,291

(22) PCT Filed: Mar. 30, 2021
(86) PCT No.: PCT/EP2021/058293

§ 371 Date: Sep. 26, 2022

Related U.S. Application Data
(60) Provisional application No. 63/004,646, filed on Apr. 3, 2020.

(51) Int. Cl. A61K 31/4985; A61K 45/06; A61P 37/06; A61P 11/00

(52) U.S. Cl. CPC A61K 31/4985; A61K 45/06; A61P 37/06; A61P 11/00

(57) ABSTRACT

This invention relates to the new use of neurokinin-1(NK-1) receptor antagonists for treating sepsis, septic shock, systemic inflammatory response syndrome (SIRS), acute respiratory distress syndrome (ARDS) or multiple organ dysfunction syndrome (MODS). The invention further relates to pharmaceutical compositions comprising NK-1 receptor antagonists and combinations with one or more therapeutic agents, for such uses.

### FIELD OF THE INVENTION

This invention relates to the new use of neurokinin-1(NK-1) receptor antagonists for treating sepsis, septic shock, systemic inflammatory response syndrome (SIRS), acute respiratory distress syndrome (ARDS) or multiple organ dysfunction syndrome (MODS) and SARS. The invention further relates to pharmaceutical compositions comprising NK-1 receptor antagonists for such uses.

### BACKGROUND OF THE INVENTION

Neurokinin-1 (NK-1; substance P) receptor antagonists are being developed for the treatment of a number of disorders associated with an excess or imbalance of tachykinins, and in particular substance P, the cognate agonist ligand of the NK-1 receptor. Examples of conditions in which substance P has been implicated include disorders of the central nervous system such as depression, and neural hypersensitivity conditions. NK-1 receptor antagonists also possess antiemetic properties and are efficacious in preventing nausea and vomiting associated with cancer chemotherapy.

An example of an NK-1 receptor antagonist which has been used in trials studying disorders of the central nervous system and neural hypersensitivity conditions is orvepitant.

Additionally, orvepitant has been found useful in the treatment of chronic cough.

Orvepitant, otherwise known as 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide has the following chemical structure (I).

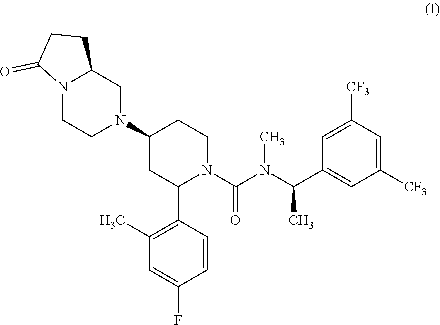 [SMILES: Cc1cc(F)ccc1C1C[C@@H](N2CCN3C(=O)CC[C@H]3C2)CCN1C(=O)N(C)[C@H](C)c1cc(C(F)(F)F)cc(C(F)(F)F)c1]

Orvepitant may also be known as:

CAS Index name

1-Piperidinecarboxamide, N-[(1R)-[3,5-bis(trifluoromethyl)phenyl]ethyl]-2-(4-fluoro-2-methylphenyl)-4-[(8aS)-hexahydro-6-oxopyrrolo[1,2-a]pyrazin-2(1H)-yl]-N-methyl-, (2R,4S)

and

IUPAC name:

(2R,4S)-N-{(1 R)-[3,5-bis(trifluoromethyl)phenyl]ethyl}-2-(4-fluoro-2-methylphenyl)-N-methyl-4-[(8aS)-6-oxohexahydropyrrolo[1,2-a]pyrazin-2(1H)-yl]-1-piperidinecarboxamide.

WO2003/066635 describes a number of diazabicycle derivatives as antagonists of tachykinin receptors, also known as substance P (SP) receptors or NK receptors and in particular NK-1 receptors, including orvepitant.

A preferred salt of the compound (I) is its hydrochloride salt which is otherwise known as orvepitant hydrochloride.

A further preferred salt of the compound (I) is its maleate salt which is otherwise known as orvepitant maleate.

WO2009/124996 describes a new crystalline form of orvepitant maleate namely anhydrous crystalline form (Form1) and pharmaceutical compositions containing it.

WO2017/118584 describes orvepitant, pharmaceutically acceptable salt and crystalline form thereof and pharmaceutical compositions containing it in the treatment of chronic cough.

A further example of an NK-1 receptor antagonist is aprepitant or its prodrug, fosaprepitant, or salts thereof.

Aprepitant (CAS number 170729-80-3) is also known by the IUPAC name 5-([(2R,3S)-2-((R)-[3,5-bis(trifluoromethyl)phenyl]ethoxy)-3-(4-fluorophenyl)morpholino]methyl)-1H-1,2,4-triazol-3(2H)-one and it can be represented by the following structural formula:

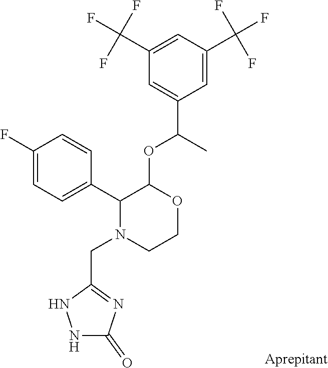 [SMILES: CC(OC1OCCN(Cc2nc(=O)[nH][nH]2)C1c1ccc(F)cc1)c1cc(C(F)(F)F)cc(C(F)(F)F)c1]

Aprepitant is used in a clinical setting for prevention of acute and delayed chemotherapy-induced nausea and vomiting (CINV) and for prevention of postoperative nausea and vomiting. Aprepitant may also be useful in the treatment of cyclic vomiting syndrome and late-stage chemotherapy-induced vomiting (CIV). Aprepitant is marketed as an oral formulation under the trade name Emend™.

Fosaprepitant (CAS number 172673-20-0), also known by the IUPAC name [3-[[(2R,3S)-2-[(1R)-1-[3,5-bis(trifluoromethyl)phenyl]ethoxy]-3-(4-fluorophenyl)morpholin-4-yl]methyl]-5-oxo-4H-1,2,4-triazol-1-yl]phosphonic acid, is a prodrug of aprepitant and it can be represented by the following structural formula. Fosaprepitant as a dimeglumine salt is an antiemetic drug administered intravenously and is commercially available under the trade name EMEND™ for injection in US and IVEMEND™ in Europe.

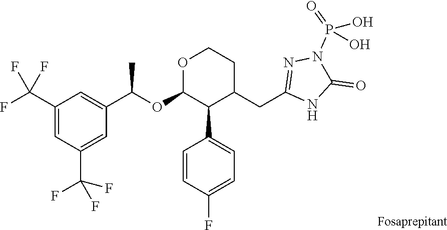 [SMILES: C[C@@H](O[C@H]1OCCC(Cc2nn(P(=O)(O)O)c(=O)[nH]2)[C@H]1c1ccc(F)cc1)c1cc(C(F)(F)F)cc(C(F)(F)F)c1]

Further examples of NK-1 receptor antagonists include for example rolapitant (trade name Varubi™), intravenous (IV) rolapitant (Varubi® IV), netupitant (when combined with palonosetron the trade name is Akynzeo®), its prodrug fosnetupitant (Akynzeo® IV; again in combination with palonosetron), serlopitant or tradipitant. They can be represented by the following structural formulae:

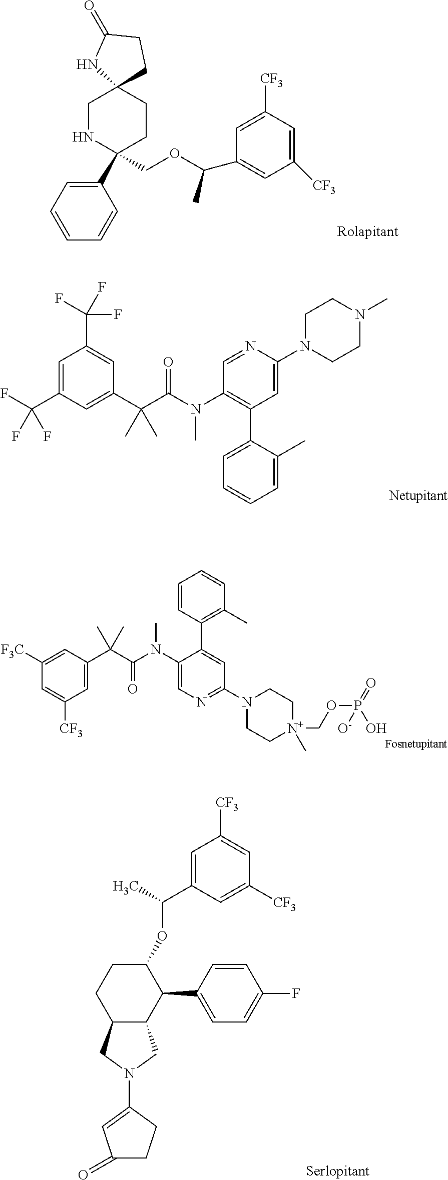 [SMILES: C[C@@H](OC[C@@]1(c2ccccc2)CC[C@]2(CCC(=O)N2)CN1)c1cc(C(F)(F)F)cc(C(F)(F)F)c1 ; Cc1ccccc1-c1cc(N2CCN(C)CC2)ncc1N(C)C(=O)C(C)(C)c1cc(C(F)(F)F)cc(C(F)(F)F)c1 ; Cc1ccccc1-c1cc(N2CC[N+](C)(COP(=O)([O-])O)CC2)ncc1N(C)C(=O)C(C)(C)c1cc(C(F)(F)F)cc(C(F)(F)F)c1 ; C[C@@H](O[C@H]1CC[C@@H]2CN(C3=CC(=O)CC3)C[C@H]2[C@@H]1c1ccc(F)cc1)c1cc(C(F)(F)F)cc(C(F)(F)F)c1]

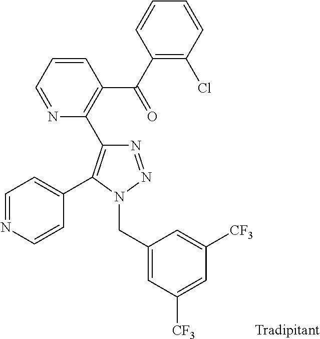 [SMILES: O=C(c1ccccc1Cl)c1cccnc1-c1nnn(Cc2cc(C(F)(F)F)cc(C(F)(F)F)c2)c1-c1ccncc1]

Rolapitant (CAS number 552292-08-7), also known by the IUPAC name (5S,8S)-8-[{(IR)-1-(3,5-Bis-(trifluoromethyl)phenyl)-ethoxy}-methyl]-8-phenyl-1,9-diazaspiro[4.5]decan-2-one, is approved by the FDA, under trade names VARUBI™ and VARUBI™IV, for oral and injectable use respectively, to prevent delayed phase chemotherapy-induced nausea and vomiting (emesis).

Netupitant (CAS number 552292-08-7), also known by the IUPAC name 2-[3,5-bis(trifluoromethyl)phenyl]-N,2-dimethyl-N-[4-(2-methylphenyl)-6-(4-methylpiperazin-1-yl)pyridine-3-yl]propanamide, is an antiemetic drug. In the United States, the combination drug netupitant/palonosetron for oral use (trade name Akynzeo™) is approved by the FDA for the prevention of acute and delayed nausea and vomiting associated with initial and repeat courses of cancer chemotherapy.

Fosnetupitant (CAS number 1703748-89-3), also known by the IUPAC name 4-(5-{2-[3,5-bis(trifluoromethyl)phenyl]-N,2-dimethylpropanamido}-4-(2-methylphenyl)pyridin-2-yl)-1-[(hydrogen phosphonatooxy)methyl]-1-methylpiperazin-1-ium, is a pro-drug of netupitant In the United States, the combination drug, fosnetupitant hydrochloride/palonosetron for intravenous use (trade name Akynzeo™ IV) is approved by the FDA for the prevention of acute and delayed nausea and vomiting associated with initial and repeat courses of highly emetogenic cancer.

Serlopitant (CAS number 860642-69-9) also known by the IUPAC name 3-[(3aR,4R,5S,7aS)-5-[(1 R)-[3,5-bis(trifluoromethyl)phenyl]ethoxy]-4-(4-fluorophenyl)-octahydro-1H-isoindol-2-yl]cyclopent-2-en-1-one has been investigated for the treatment of chronic itch conditions including Prurigo Nodularis.

Tradipitant (CAS number 622370-35-8) also known by the IUPAC name 2-(1-{([3,5-bis(trifluoromethyl)phenyl]methyl}-5-(pyridin-4-yl)-1H-1,2,3-triazol-4-yl)-3-(2-chlorobenzoyl)pyridine, has been used in trials studying the treatment of atopic dermatitis, gastroparesis, and motion sickness, among others.

We have now surprisingly found that NK-1 receptor antagonists are also useful in treating sepsis, septic shock, systemic inflammatory response syndrome (SIRS), acute respiratory distress syndrome (ARDS) or multiple organ dysfunction syndrome (MODS).

Sepsis is a life-threatening condition that arises due to a dysregulated host response to infection, that causes the body to injure its own tissues and organs (Singer et al., 2016); this includes puerperal sepsis. If sepsis is not recognized early and managed promptly, it can lead to septic shock, ARDS, multiple organ dysfunction syndrome (MODS) and ultimately death. Any type of infectious pathogen can cause sepsis including coronaviruses. Sepsis is estimated to affect more than 30 million people globally every year, with over 5 million deaths (Fleischmann et al., 2016). Septic shock occurs in a subset of sepsis patients in which particularly profound circulatory, cellular, and metabolic abnormalities are associated with a greater risk of mortality than with sepsis alone (Singer et al., 2016). Septic shock is characterised by dangerous and persistent low blood pressure. In the absence of infection, a sepsis-like disorder may manifest in patients termed systemic inflammatory response syndrome (SIRS). This exaggerated host response of the body to noxious stressors can be due to trauma, surgery, aspiration, ventilation, acute inflammation, ischemia or reperfusion, or malignancy (Balk, 2014). Sepsis, septic shock and SIRS, can ultimately progress to ARDS and MODS.

Respiratory failure due to acute respiratory distress syndrome (ARDS) is a life-threatening condition where the lungs become severely inflamed and cannot provide the body's vital organs with sufficient oxygen (Matthay et al., 2019). Patients require supportive airway ventilation, moreover the mortality rate is extremely high and some patients experience long term pulmonary dysfunction. ARDS can be caused by viral infections with coronaviruses such as COVID-19 (Wujtewicz et al., 2020), where it is called COVID-19-associated ARDS (CARDS) (Kenny, 2020). Other frequent causes are bacterial infection, non-pulmonary sepsis, aspiration of gastric and/or oral and oesophageal contents and major trauma (such as blunt or penetrating injuries or burns) (Matthay et al., 2019). Approximately 200,000 patients are affected by ARDS in the US each year resulting in 75,000 deaths; globally there are over 3 million cases annually (Fan et al., 2018).

Severity of ARDS is defined on the degree of hypoxemia suffered by patients (ARDS Definition Task Force, 2012). ARDS pathology shows diffuse alveolar damage (DAD) in the lungs, plus alveolar epithelial and lung endothelial injury that results in accumulation of protein-rich inflammatory oedematous fluid in the alveolar space. This early inflammatory phase is followed by a fibroproliferative repair phase, leading to either the resolution of ARDS or irreversible lung fibrosis (Ware & Matthay, 2000). The invasive mechanical ventilation so necessary as a life-supporting therapy for ARDS patients in the acute phase (Cabrera-Benitez et al., 2014) is also a major contributor to a fibroproliferative response.

Patients who survive ARDS may also experience significant reductions in health-related quality-of-life (QoL) that may last for many years following hospital discharge (Davidson et al., 1999; Chiumello et al., 2018; Bein et al., 2018). In health surveys, ARDS survivors had clinically significant reductions in their mental health, physical functioning, social functioning, vitality and in pulmonary disease-specific QoL domains, including dyspnoea (Davidson et al., 1999). There are no approved ARDS pharmacotherapies.

MODS is the development of potentially reversible physiologic derangement involving two or more organ systems not involved in the disorder that results in intensive care unit (ICU) admission and arising in the wake of a potentially life-threatening physiologic insult (Marshall, 2001). An unbalanced immune response as a result of sepsis or SIRS can progress to MODS. Lungs are most often the first organ initiating the MODS cascade. Other organs/systems affected are the cardiovascular, gastrointestinal, liver, haematologic (including coagulation), immune, metabolic and endocrine. MODS can also be due to sepsis of the foetus or new-born. ARDS fatality rates alone are 40-50%; once additional organ system dysfunction occurs this increases to as high as 90%. Clinical trials have shown fatality rates ranging from 40% to 75% in MODS that result from sepsis (Al-Khafaji, 2020). These critically ill patients require aggressive treatment in an ICU setting to prevent death.

### SUMMARY OF THE INVENTION

The solution provided by the present invention is the use of NK-1 receptor antagonists in treating sepsis, septic shock, systemic inflammatory response syndrome (SIRS), acute respiratory distress syndrome (ARDS) or multiple organ dysfunction syndrome (MODS).

Thus, in one aspect, this invention provides a method of treating sepsis, septic shock, systemic inflammatory response syndrome (SIRS), acute respiratory distress syndrome (ARDS) or multiple organ dysfunction syndrome (MODS) in a patient in need of such treatment comprising administering to said patient a therapeutically effective amount of NK-1 receptor antagonist.

In a further aspect thereof, the invention provides NK-1 receptor antagonists for use in treating sepsis, septic shock, systemic inflammatory response syndrome (SIRS), acute respiratory distress syndrome (ARDS) or multiple organ dysfunction syndrome (MODS).

In a yet further aspect thereof, the invention provides the use of NK-1 receptor antagonists for the manufacture of a medicament for treating sepsis, septic shock, systemic inflammatory response syndrome (SIRS), acute respiratory distress syndrome (ARDS) or multiple organ dysfunction syndrome (MODS).

In a further aspect, the invention provides a method of treating sepsis, septic shock, acute respiratory distress syndrome (ARDS) or multiple organ dysfunction syndrome (MODS), due to or associated with coronavirus infection in a patient in need of such treatment comprising administering to said patient a therapeutically effective amount of NK-1 receptor antagonist.

In another aspect the invention provides a pharmaceutical composition comprising an NK-1 receptor antagonist and more pharmaceutically acceptable carriers or excipients for treating sepsis, septic shock, systemic inflammatory response syndrome (SIRS), acute respiratory distress syndrome (ARDS) or multiple organ dysfunction syndrome (MODS).

In a further aspect, the invention provides a method of treating acute respiratory distress syndrome (ARDS) due to or associated with acute exacerbations of interstitial lung diseases (AE-ILD) including idiopathic pulmonary fibrosis (AE-IPF) in a patient in need of such treatment comprising administering to said patient a therapeutically effective amount of an NK-1 receptor antagonist.

In a further aspect, the invention provides a method of treating acute respiratory distress syndrome (ARDS) due to or associated with a coronavirus infection in a patient in need of such treatment comprising administering to said patient a therapeutically effective amount of an NK-1 receptor antagonist.

In a further aspect, the invention provides a method of treating acute respiratory distress syndrome (ARDS) due to or associated with a COVID-19 infection (COVID19-associated ARDS [CARDS]) in a patient in need of such treatment comprising administering to said patient a therapeutically effective amount of an NK-1 receptor antagonist.

### BRIEF DESCRIPTION OF THE DRAWINGS

FIG. 1 shows the effect of orvepitant (dose of 250 mg/kg of orvepitant maleate anhydrous crystalline form [Form 1] administered per os) compared to control groups on inflammatory cell counts in BALF following LPS intranasal challenge at 24 hours. A). Total white blood cell (WBC) counts. B) Neutrophil cell counts. Statistical analysis was performed by One Way ANOVA followed by Dunnetts's multiple comparison test, all groups vs vehicle+LPS-challenge treated group, n=10 for each group. Data are shown as mean±standard error of mean (SEM), ***p<0.001.

Key: Bronchoalveolar lavage fluid (BALF); intranasal (in); lipopolysaccharide (LPS); number of animals (n); phosphate buffered saline (PBS); white blood cells (WBC).

### DETAILED DESCRIPTION OF THE INVENTION

While various embodiments of the present disclosure are described herein, it will be obvious to those skilled in the art that such embodiments are provided by way of example only. Numerous modifications and changes to and variations and substitutions of the embodiments described herein will be apparent to those skilled in the art without departing from the disclosure. It is understood that various alternatives to the embodiments described herein may be employed in practicing the disclosure. It is also understood that every embodiment of the disclosure may optionally be combined with any one or more of the other embodiments described herein which are consistent with that embodiment.

It is further understood that, in general, where an embodiment in the description or the claims is referred to as comprising one or more features, the disclosure also encompasses embodiments that consist of, or consist essentially of, such feature(s).

It is further understood that the present disclosure encompasses analogs, derivatives, prodrugs, metabolites, salts, solvates, hydrates, clathrates and polymorphs of all of the compounds/substances disclosed herein, as appropriate. The specific recitation of “analogs”, “derivatives”, “prodrugs”, “metabolites”, “salts”, “solvates”, “hydrates”, or “polymorphs” with respect to a compound/substance or a group of compounds/substances in certain instances of the disclosure shall not be interpreted as an intended omission of any of these forms in other instances of the disclosure where the compound/substance or the group of compounds/substances is mentioned without recitation of any of these forms.

All patent literature and all non-patent literature cited herein are incorporated herein by reference in their entirety to the same extent as if each patent literature or non-patent literature were specifically and individually indicated to be incorporated herein by reference in its entirety.

Definitions

Unless defined otherwise or indicated otherwise by their use herein, all technical and scientific terms used herein have the same meaning as commonly understood by one of ordinary skill in the art to which this application belongs.

All numbers expressing quantities, percentages or proportions, and other numerical values used in the specification and claims, are to be understood as being modified in all instances by the term “about.”

It should be understood that the terms “a” and “an” as used herein refer to “one or more” of the enumerated components. It will be clear to one of ordinary skill in the art that the use of the singular includes the plural unless specifically stated otherwise.

As used herein, the terms “treatment,” “treating,” and the like, refer to obtaining a desired pharmacologic, physiologic, dermatologic or cosmetic effect. The effect may be prophylactic in terms of completely or partially preventing a condition or disease or disorder or symptom thereof and/or may be therapeutic in terms of a partial or complete cure for a condition or disease or disorder and/or adverse symptom or effect attributable to the condition or disease or disorder. It will be appreciated that the effect attributable to the condition or disease or disorder includes the longer-term sequelae of the disorder and/or adverse symptom or effect attributable to the condition or disease or disorder. Reference to “treatment” of a medical condition includes preventing (precluding), reducing the risk of developing, delaying the onset of, and slowing the progression of, the condition or one or more symptoms or complications associated with the condition.

The terms “treatment,” “treating,” and the like also mean prolonging survival as compared to expected survival if not receiving treatment, improving quality of life, and reducing health care costs and utilisation.

The terms “treatment,” “treating,” and the like also means an antimicrobial effect that kills or prevents or inhibits the grow of microorganisms thereby acting as antibiotics, antifungals, antiprotozoals, and antivirals.

The terms “treatment,” “treating,” in the context of a coronavirus infection such as COVID-19, means killing or preventing or supressing the ability of the virus to enter into host cells and to replicate and thereby reduces viral load.

“Treatment,” thus, for example, covers any treatment of a condition or disease in a mammal, particularly in a human, and includes: (a) preventing the condition or disease, disorder or symptom thereof from occurring in a subject which may be predisposed to the condition or disease or disorder but has not yet been diagnosed as having it; (b) inhibiting the condition or disease, disorder or symptom thereof, such as, arresting its development; and (c) relieving, alleviating or ameliorating the condition or disease or disorder or symptom thereof, such as, for example, causing regression of the condition or disease or disorder or symptom thereof.

As used herein, the term “effective amount” means that amount of a drug or pharmaceutical agent that will elicit the biological or medical response of a tissue, system, animal or human that is being sought, for instance, by a researcher, clinician or veterinarian.

The term “NK-1 receptor” refers to a member of the G protein-coupled superfamily of receptors called tachykinin receptors. The tachykinins, also called neurokinins, are a family of peptide neurotransmitters that mediate the release of intracellular calcium by binding to a group of transmembrane receptors called neurokinin (NK) receptors. Mammalian tachykinin receptors consist of three types: Neurokinin-1 (NK-1), Neurokinin-2 (NK-2), and Neurokinin-3 (NK-3) receptors. Substance P(SP) is the cognate agonist ligand of the NK-1 receptor which is also known as the SP receptor, though other tachykinins can bind the NK-1 receptor with lower affinity. Antagonists of the NK-1 receptor, are thus of use in the treatment of conditions mediated by tachykinins, in particular SP.

The term “ pharmaceutically acceptable” refers to those compounds (or salts, prodrugs, tautomers, zwitterionic forms, etc.) which are suitable for use in contact with the tissues of patients without excessive toxicity, irritation, allergic response, immunogenicity, are commensurate with a reasonable benefit/risk ratio, and are effective for their intended use.

As used herein, “pharmaceutically acceptable excipient” or “pharmaceutically acceptable carrier” mean a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound according to the invention when administered to a patient and interactions which would result in pharmaceutical compositions that are not pharmaceutically acceptable are avoided. In addition, each excipient must of course be pharmaceutically acceptable e.g. of sufficiently high purity.

The term “therapeutically effective amount” refers to an amount of a substance that, when administered to a subject, is sufficient to prevent reduce the risk of developing, delay the onset of, or slow the progression of the medical condition being treated, or to alleviate to some extent one or more symptoms or complications of that condition. The term “therapeutically effective amount” also refers to an amount of a substance that is sufficient to elicit the biological or medical response of a cell, tissue, organ, system, animal or human which is sought by a researcher, veterinarian, medical doctor or clinician.

The term “subject” refers to an animal, including a mammal, such as a primate (e.g., a human, a chimpanzee or a monkey), a rodent (e.g., a rat, a mouse, a guinea pig, a gerbil or a hamster), a lagomorph (e.g., a rabbit), a swine (e.g., a pig), an equine (e.g., a horse), a canine (e.g., a dog) or a feline (e.g., a cat).

The terms “subject” and “patient” are used interchangeably herein in reference, e.g., to a mammalian subject, such as a human subject.

The term sepsis and septicaemia are intended to have the same meaning and are used interchangeably herein.

The term acute respiratory distress syndrome (ARDS) and acute lung injury (ALI) are intended to have the same meaning and are used interchangeably herein.

The terms multiple organ dysfunction syndrome (MODS), multiple organ dysfunction (MOD), multiple organ failure (MOF), total organ failure (TOF) or multisystem organ failure (MSOF) or acute organ dysfunction are intended to have the same meaning are used interchangeably herein.

The term “about” or “approximately” means an acceptable error for a particular value as determined by one of ordinary skill in the art, which depends in part on how the value is measured or determined.

The term COVID-19 associated ARDS (CARDS) refers to development of ARDS following COVID-19 infection.

COVID-19 associated ARDS and COVID associated ARDS are intended to have the same meaning and are used interchangeably herein.

The term acute exacerbation of interstitial lung diseases (AE-ILD) refers to interstitial lung diseases (ILD), wherein the patients suffering from ILD develops a rapid and significant decline in pulmonary function.

The term acute exacerbation of idiopathic pulmonary fibrosis (AE-IPF) refers to idiopathic pulmonary fibrosis (IPF) disease, wherein the patients suffering from IPF develop a rapid and significant decline in pulmonary function.

In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 20%, 15′%, 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

The term coronavirus as used herein refers to human pathogenic coronaviruses including severe acute respiratory syndrome coronavirus (SARS-CoV) (Coleman & Frieman, 2014), Middle East respiratory syndrome coronavirus (MERS-CoV) (Coleman & Frieman, 2014) and SARS-CoV-2 (World Health Organisation, Interim Guidance, 2020).

The coronavirus SARS-CoV and SARS are intended to have the same meaning and are used interchangeably herein.

The coronavirus MERS-CoV and MERS are intended to have the same meaning and are used interchangeably herein.

The coronavirus SARS-CoV-2, COVID-19, and 2019-nCoV are intended to have the same meaning and are used interchangeably herein.

Sepsis and septic shock are defined by ‘The Third International Consensus Definitions for Sepsis and Septic Shock (Sepsis-3)’ (Singer et al., 2016).

MODS is defined as “the development of potentially reversible physiologic derangement involving two or more organ systems not involved in the disorder that results in ICU admission and arising in the wake of a potentially life-threatening physiologic insult' (Marshall, 2001).

The term “medical conditions” (or “conditions” for short) encompasses disorders and diseases.

The term “respiratory failure” refers to a condition resulting from inadequate gas exchange by the respiratory system in which insufficient oxygen passes from the lungs into blood and insufficient CO2 is expelled from the body.

The term “combination” as used herein refers to either a fixed combination in one dosage unit form, or non-fixed combination.

The term “fixed combination” means that the active ingredients, e.g. a compound of formula (I)-(IX) or pharmaceutically acceptable salt thereof and a combination partner, are both administered to a patient simultaneously in the form of a single entity or dosage.

The term “non-fixed combination” means that the active ingredients, e.g. a compound (I)-(IX) or pharmaceutically acceptable salt thereof and a combination partner, (e.g. another drug as explained below, also referred to as “therapeutic agent” or “co-agent”) are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

The terms “co-administration” or “combined administration” or the like as utilized herein are meant to encompass administration of the compound (I) and the selected combination partner to a single subject in need thereof (e.g. a patient), and are intended to include treatment regimens in which the agents are not necessarily administered by the same route of administration or at the same time.

The term “prodrug” is meant a compound that, after administration, is converted within the body into a pharmacologically active drug.

The term “metabolites” means the intermediate end product of metabolism, formed as part of the natural biochemical process of degrading and eliminating the compounds.

Available evidence supports a pathological role of Substance P (SP) in the development of sepsis/septic shock, SIRS, ARDS and MODS.

SP is the preferred ligand of the NK-1 receptor among the three tachykinin receptors NK-1, NK-2 and NK-3.

SP acting on the NK-1 receptor system could be a major contributor to the uncontrolled inflammatory response of the medical conditions herein and later fibroproliferative phase that can lead to long term pulmonary dysfunction and lowered QoL characteristic of ARDS.

By inhibiting NK-1 or blocking binding of substance P to the NK-1 receptor, an NK-1 receptor antagonist could alleviate the unabated inflammatory response that drives the progression of sepsis, septic shock or SIRS to ARDs or/and MODS by SP.

Use of an NK-1 receptor antagonist could prevent or reduce the incidence and intensity of inflammatory response associated with medical conditions described herein.

The present invention provides a method of treatment of sepsis, septic shock, systemic inflammatory response syndrome (SIRS), acute respiratory distress syndrome (ARDS) or multiple organ dysfunction syndrome (MODS) in a patient in need of such treatment comprising administering to said patient a therapeutically effective amount of NK-1 receptor antagonist.

In some embodiments, the present invention provides a method of treatment of acute respiratory distress syndrome (ARDS).

In some embodiments, the present invention provides a method of treatment of acute exacerbation of interstitial lung diseases (AE-ILD) and acute exacerbation of idiopathic pulmonary fibrosis (AE-IPF) in a patient in need of such treatment comprising administering to said patient a therapeutically effective amount of NK-1 receptor antagonist.

In some embodiments, the present invention provides a method of treatment of acute exacerbation of interstitial lung diseases (AE-ILD) and acute exacerbation of idiopathic pulmonary fibrosis (AE-IPF) in a patient in need of such treatment comprising administering to said patient a therapeutically effective amount of an NK-1 receptor antagonist, wherein the NK-1 receptor antagonist is selected from orvepitant, aprepitant, fosaprepitant, rolapitant, netupitant, fosnetupitant, serlopitant, tradipitant or prodrug, metabolites or pharmaceutically acceptable salt thereof.

In some embodiments the acute respiratory distress syndrome (ARDS) is due to or associated with acute exacerbations of interstitial lung diseases (AE-ILD) including idiopathic pulmonary fibrosis (AE-IPF).

In some embodiments the sepsis, septic shock, acute respiratory distress syndrome (ARDS) or multiple organ dysfunction syndrome (MODS) are due to or associated with coronavirus infections.

In certain embodiments the sepsis, septic shock, acute respiratory distress syndrome (ARDS) or multiple organ dysfunction syndrome (MODS) are due to or associated with COVI D-19 infections.

In some embodiments the acute respiratory distress syndrome (ARDS) is due to or associated with coronavirus infections, particularly COVID-19 infections.

Surviving ARDS is associated with a substantial long-term reduction in health-related quality of life. Particularly, survival of ARDS is often associated with sequelae of mental, physical and pulmonary dysfunction, and vitality and social impairments after hospital discharge.

Mental impairments include for example anxiety, depression, posttraumatic stress disorder symptoms.

Physical impairments include, for example, fatigue, muscle weakness, reduced physical status, impairment of activities of daily life and reduced walking capacity.

Pulmonary dysfunction impairments include, for example, dyspnoea (shortness of breath) and reduced exercise capacity.

Social impairments include, for example, the ability to participate in social roles and activities.

In some embodiments, the invention provides a method of treatment the longer-term sequelae of the disorder and/or adverse symptoms or effect attributable to ARDS.

NK-1 Receptor Antagonists

One or more NK-1 receptor antagonists can be used to treat of treatment sepsis, septic shock, systemic inflammatory response syndrome (SIRS), acute respiratory distress syndrome (ARDS) or multiple organ dysfunction syndrome (MODS).

In some embodiments, the NK-1 receptor antagonist is or comprises a selective NK-1 receptor antagonist.

Non-limiting examples of NK-1 receptor antagonists include orvepitant, aprepitant, fosaprepitant, rolapitant, netupitant, fosnetupitant, serlopitant, tradipitant or analogs, derivatives, prodrug, metabolites or pharmaceutically acceptable salt thereof.

In some embodiments the sepsis, septic shock, acute respiratory distress syndrome (ARDS) or multiple organ dysfunction syndrome (MODS) are due to or associated with coronavirus infection and are treated with an NK-1 receptor antagonist selected from orvepitant, aprepitant, fosaprepitant, rolapitant, netupitant, fosnetupitant, serlopitant, tradipitant or prodrug , metabolites or pharmaceutically acceptable salt thereof.

In certain embodiments, the NK-1 receptor antagonist is or comprises orvepitant or pharmaceutically acceptable salt, solvate, hydrate, or metabolite thereof.

Chemically, the generic name orvepitant refers to Compound (I).

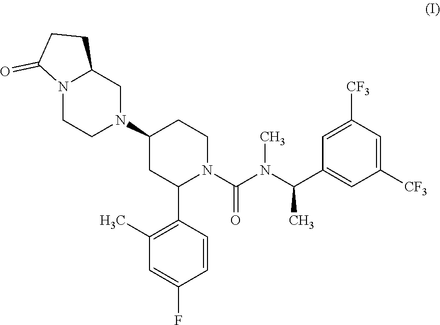 [SMILES: Cc1cc(F)ccc1C1C[C@@H](N2CCN3C(=O)CC[C@H]3C2)CCN1C(=O)N(C)[C@H](C)c1cc(C(F)(F)F)cc(C(F)(F)F)c1]

The compound (I) or its pharmaceutically acceptable salts may be prepared by the processes described in PCT Publication Nos. WO2003/066635, WO2009/124996, WO2007/048642 and WO2017/118584 which are incorporated herein by reference.

Specifically, the Examples 9a and 11 of WO2003/066635 describe the synthesis of the compound (I) as free base and as hydrochloride salt respectively. Specific crystalline forms of hydrochloride salt namely anhydrous and dihydrate crystalline forms are described in the Examples 11a and 11b respectively. Example 11c describes the synthesis of the compound (I) as a maleate salt. Examples 2-8 of WO2009/124996 describe the synthesis of the maleate salt of the compound (I) as anhydrous crystalline form (Form1).

Orvepitant maleate Form 1 is characterized by X-ray powder diffraction (XRD) pattern expressed in terms of 2 theta angles and obtained with a diffractometer using copper KαX-radiation, wherein the XRD pattern comprises 2 theta angle peaks at essentially at 7.3±0.1, 7.5±0.1, 10.9±0.1, 12.7±0.1, 16.5±0.1 degrees, which correspond respectively to d-spacings at 12.2, 11.8, 8.1, 7.0 and 5.4 Angstroms (Å).

Example 1 of WO2007/048642 discloses a process for preparing an intermediate in the synthesis of the compound (I).

In some embodiments the compound for use according to the present invention is or comprise orvepitant maleate.

In other embodiments the compound for use according to the present invention is or comprise orvepitant maleate as anhydrous crystalline form.

In further embodiments the compound for use according to the present invention is or comprise maleate as anhydrous crystalline Form 1.

In one embodiment, the present invention provides orvepitant or pharmaceutically acceptable salts thereof for use in the treatment of ARDS.

In another embodiment, the present invention provides orvepitant maleate for use in the treatment of ARDS.

In another embodiment, the present invention provides orvepitant maleate as anhydrous crystalline Form 1 for use in the treatment of ARDS.

In one embodiment, the present invention provides orvepitant or pharmaceutically acceptable salts thereof for use in the treatment of ARDS, wherein the ARDS is due to or associated with a coronavirus infection.

In another embodiment, the present invention provides orvepitant maleate for use in the treatment of ARDS, wherein the ARDS is due to or associated with a coronavirus infection.

In another embodiment, the present invention provides orvepitant maleate as anhydrous crystalline Form 1 for use in the treatment of ARDS, wherein the ARDS is due to or associated with a coronavirus infection.

In one embodiment, the present invention provides orvepitant or pharmaceutically acceptable salts thereof for use in the treatment of ARDS, wherein the ARDS is due to or associated with acute exacerbations of interstitial lung diseases (AE-ILD).

In one embodiment, the present invention provides orvepitant or pharmaceutically acceptable salts thereof for use in the treatment of ARDS, wherein the ARDS is due to or associated with acute exacerbations of idiopathic pulmonary fibrosis (AE-IPF).

In another embodiment, the present invention provides orvepitant maleate for use in the treatment of ARDS, wherein the ARDS is due to or associated with acute exacerbations of interstitial lung diseases (AE-ILD).

In another embodiment, the present invention provides orvepitant maleate for use in the treatment of ARDS, wherein the ARDS is due to or associated with acute exacerbations of idiopathic pulmonary fibrosis (AE-IPF).

In another embodiment, the present invention provides orvepitant maleate as anhydrous crystalline Form 1 for use in the treatment of ARDS, wherein the ARDS is due to or associated with acute exacerbations of interstitial lung diseases (AE-ILD).

In another embodiment, the present invention provides orvepitant maleate as anhydrous crystalline Form 1 for use in the treatment of ARDS, wherein the ARDS is due to or associated with acute exacerbations of idiopathic pulmonary fibrosis (AE-IPF).

In additional embodiments the NK-1 receptor antagonist is or includes aprepitant or fosaprepitant (a prodrug of aprepitant) or a pharmaceutically acceptable salt, solvate, hydrate, clathrate, polymorph prodrug or metabolite thereof.

Chemically the generic name of aprepitant refers to Compound (II).

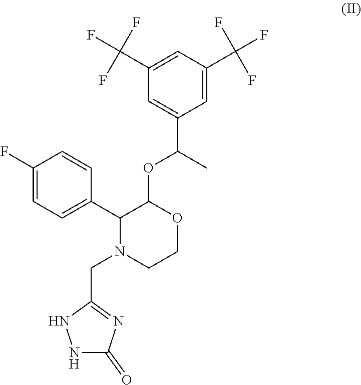 [SMILES: CC(OC1OCCN(Cc2nc(=O)[nH][nH]2)C1c1ccc(F)cc1)c1cc(C(F)(F)F)cc(C(F)(F)F)c1]

The compound (II) or its pharmaceutically acceptable salts may be prepared by the processes described in PCT Publication No. WO94/00440 and WO95/16679 which are incorporated herein by reference. Specifically, Example 75 of PCT Publication No. WO95/16679 describes the synthesis of compound (II).

Polymorphic forms of compound of formula (II) may be prepared by the processes described in U.S. Pat. No. 6,096,742 which is also incorporated herein by reference.

Specifically, a polymorphic forms of the compound (II) characterized by an X-ray powder diffraction pattern comprising 2 theta angle peaks at essentially at 12.0, 15.3, 16.6, 17.0, 17.6, 19.4, 20.0, 21.9, 23.6, 23.8 is described in U.S. Pat. No. 6,096,742.

In some embodiments, the compound for use according to the present invention is or comprise aprepitant or a pharmaceutically acceptable salt thereof.

In some embodiments, the compound for use according to the present invention is or comprise aprepitant as a crystalline form.

Chemically, the generic name of fosaprepitant refers to compound of formula (III).

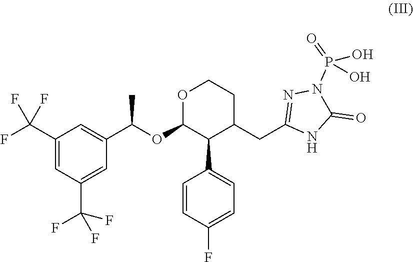 [SMILES: C[C@@H](O[C@H]1OCCC(Cc2nn(P(=O)(O)O)c(=O)[nH]2)[C@H]1c1ccc(F)cc1)c1cc(C(F)(F)F)cc(C(F)(F)F)c1]

The compound (III) and pharmaceutically acceptable salts, including its dimeglumine salt, can be prepared by the process described in U.S. Pat. No. 5,691,336 and PCT Publication Nos. WO2010/018595 and WO2011104581, which are incorporated herein by reference.

In additional embodiments the compound for use according to the present invention is fosaprepitant or pharmaceutically acceptable salts thereof.

In further embodiments the compound for use according to the present invention is fosaprepitant dimeglumine.

In yet further embodiments the NK-1 receptor antagonist is or includes rolapitant or a pharmaceutically acceptable salt, solvate, hydrate, polymorph, produg or metabolite thereof

Chemically, the generic name of rolapitant refers to Compound (IV).

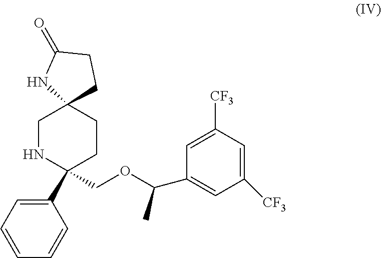 [SMILES: C[C@@H](OC[C@@]1(c2ccccc2)CC[C@]2(CCC(=O)N2)CN1)c1cc(C(F)(F)F)cc(C(F)(F)F)c1]

The compound (IV) or its pharmaceutically acceptable salts may be prepared by the processes described in U.S. Pat. No. 7,049,320, U.S. Patent Application No. 2007/0244142 and PCT Publication No. WO2005/063243, which are herein incorporated by reference.

Process for preparing pharmaceutical compositions for intravenous administration of compound (IV) or pharmaceutically acceptable salts, hydrates or prodrugs are described in U.S. Pat. No. 9,101,615, which is also incorporated by reference.

In still further embodiments, the NK-1 receptor antagonist is or includes netupitant or a pharmaceutically acceptable salt, solvate, hydrate, polymorph, prodrug or metabolite thereof.

Chemically the generic name of netupitant refers to Compound (V).

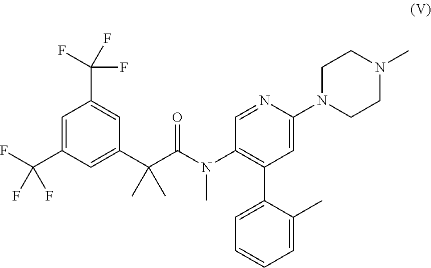 [SMILES: Cc1ccccc1-c1cc(N2CCN(C)CC2)ncc1N(C)C(=O)C(C)(C)c1cc(C(F)(F)F)cc(C(F)(F)F)c1]

The compound (V) or its pharmaceutically acceptable salts may be prepared by the processes described in U.S. Pat. No. 6,297,375 and PCT Publication No. WO2015/171489, which are herein incorporated by reference.

In other embodiments, the NK-1 receptor antagonist is or includes fosnetupitant (prodrug of netupitant) pharmaceutically acceptable salt, solvate, hydrate, clathrate, polymorph, or metabolite thereof.

Chemically, the generic name of fosnetupitant refers to Compound (VI).

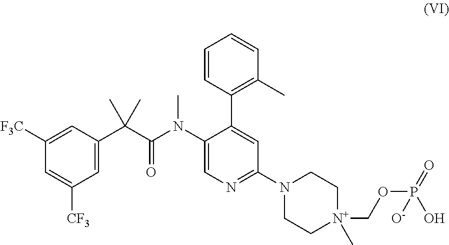 [SMILES: Cc1ccccc1-c1cc(N2CC[N+](C)(COP(=O)([O-])O)CC2)ncc1N(C)C(=O)C(C)(C)c1cc(C(F)(F)F)cc(C(F)(F)F)c1]

The compound (VI) or its pharmaceutically acceptable salts, including the hydrochloride salt, may be prepared by the processes described in U.S. Pat. No. 10,208,073, which is incorporated herein by reference.

In further embodiments the compound for use according to the present invention is fosnetupitant hydrochloride.

In yet other embodiments the NK-1 receptor antagonist is serlopitant, or a pharmaceutically acceptable salt. solvate, hydrate, clathrate, polymorph. prodrug or metabolite thereof.

Chemically, the generic name of serlopitant refers to Compound (VII).

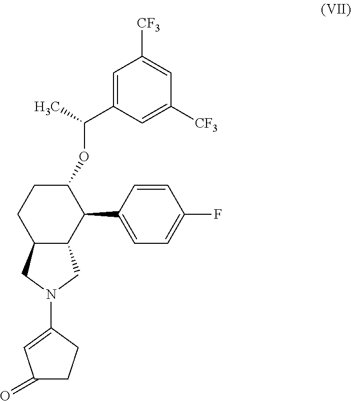 [SMILES: C[C@@H](O[C@H]1CC[C@@H]2CN(C3=CC(=O)CC3)C[C@H]2[C@@H]1c1ccc(F)cc1)c1cc(C(F)(F)F)cc(C(F)(F)F)c1]

The compound (VII) or its pharmaceutically acceptable salts may be prepared by the processes described in U.S. Pat. No. 7,217,731 and in PCT Publication No. WO2008054690, which are incorporated herein by reference.

In still other embodiments, the NK-1 receptor antagonist is or includes tradipitant, or a pharmaceutically acceptable salt. solvate, hydrate, polymorph, prodrug or metabolite thereof.

Chemically, the generic name of tradipitant refers to Compound (VIII).

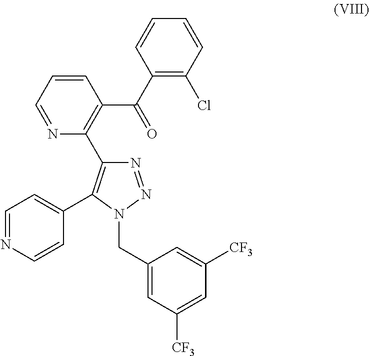 [SMILES: O=C(c1ccccc1Cl)c1cccnc1-c1nnn(Cc2cc(C(F)(F)F)cc(C(F)(F)F)c2)c1-c1ccncc1]

The compound (VIII) or its pharmaceutically acceptable salts may be prepared by the processes described in U.S. Pat. No. 7,320,994, which is incorporated herein by reference.

Pharmaceutical Compositions

In another aspect, the invention provides a pharmaceutical composition comprising an NK-1 receptor antagonist and one or more pharmaceutically acceptable carriers or excipients for use in sepsis, septic shock, systemic inflammatory response syndrome (SIRS), acute respiratory distress syndrome (ARDS) or multiple organ dysfunction syndrome (MODS).

In certain embodiments, the NK-1 receptor antagonist is selected from orvepitant, aprepitant, fosaprepitant, rolapitant, netupitant, fosnetupitant or serlopitant or a pharmaceutically acceptable salt. solvate, hydrate, polymorph, prodrug or metabolite thereof.

In additional embodiments, the NK-1 receptor antagonist is orvepitant or a pharmaceutically acceptable salt, solvate, hydrate, polymorph, prodrug or metabolite thereof.

In further embodiments, the NK-1 receptor antagonist is orvepitant maleate.

In still further embodiments, the NK-1 receptor antagonist is orvepitant maleate anhydrous crystalline form (Form 1).

Pharmaceutical compositions for use in accordance with the present invention may be formulated in a conventional manner for use in human and veterinary medicine using one or more pharmaceutically acceptable carriers or excipients.

Thus, the NK-1 receptor antagonist (e.g., orvepitant) can be administered via any suitable route including oral, buccal, sub-lingual, parenteral (including intramuscular, subcutaneous, intradermal intravascular, intravenous, intraarterial, intramedullary and intrathecal), topical (including ophthalmic and nasal), depot or rectal administration or in a form suitable for administration by inhalation or insufflation (either through the mouth or nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate or croscarmellose sodium); or wetting agents (e.g. sodium lauryl sulphate).

The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal or sub-lingual administration the composition may take the form of tablets or wafers formulated in conventional manner.

The NK-1 receptor antagonist (e.g., orvepitant) may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The NK-1 receptor antagonist (e.g., orvepitant) can be formulated for dermal administration.

Dermal administration may include topical application or transdermal administration. Transdermal application can be accomplished by suitable patches, emulsions, ointments, solutions, suspensions, pastes, foams, aerosols, lotions, creams or gels as is generally known in the art, specifically designed for the transdermal delivery of active agents, optionally in the presence of specific permeability enhancers. Topical compositions can likewise take one or more of these forms. One or more active compounds may be present in association with one or more non-toxic pharmaceutically acceptable auxiliaries such as excipients, adjuvants (e.g. buffers), carriers, inert solid diluents, suspending agents, preservatives, fillers, stabilizers, anti-oxidants, food additives, bioavailability enhancers, coating materials, granulating and disintegrating agents, binding agents etc., and, if desired, other active ingredients.

The pharmaceutical composition may be formulated, for example, for immediate release, sustained release, pulsed release, two or more step release, or depot or any other kind of release.

The manufacture of the pharmaceutical compositions according to the present subject matter may be performed according to methods known in the art and will be explained in further detail below. Commonly known and used pharmaceutically acceptable auxiliaries as well as further suitable diluents, flavourings, sweetening agents, colouring agents etc. may be used, depending on the intended mode of administration as well as particular characteristics of the active compound to be used, such as solubility, bioavailability etc.

Any non-toxic, inert, and effective topical, oral, etc. pharmaceutically acceptable carrier may be used to formulate the compositions described herein. Well-known carriers used to formulate other topical therapeutic compositions for administration to humans are useful in these compositions. Examples of these components that are well known to those of skill in the art are described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, January 1996, the contents of which are hereby incorporated by reference in their entirety. Examples of such useful cosmetically acceptable excipients, carriers and diluents include distilled water, physiological saline, Ringer's solution, dextrose solution, Hank's solution, and DMSO, which are among those suitable for use herein.

These additional other inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 17th Ed., Mack Publishing Co., Easton, Pa. (1990), both of which are incorporated by reference herein in their entirety.

In certain embodiments, the present topical compositions are formulated in a serum, a gel cream, a lotion, a cream, an ointment, a gel, an aerosol, a foam, a foamable liquid, a solution (solubilized system), a paste, a suspension, a dispersion, an emulsion, a skin cleanser, a milk, a mask, a solid stick, a bar (such as a soap bar), an encapsulated formulation, a microencapsulated formulation, microspheres or nanospheres or vesicular dispersions, or other cosmetically acceptable topical dosage form. In the case of vesicular dispersions, the vesicles may be composed of lipids, which can be of the ionic or nonionic type, or a mixture thereof.

The formulation can comprise one or more of an aqueous formulation and/or an anhydrous formulation.

In certain embodiments, the NK-1 receptor antagonist (e.g., orvepitant) is administered orally (e.g., as a capsule or tablet, optionally with an enteric coating).

In other embodiments, the NK-1 receptor antagonist (e.g., orvepitant) is administered parenterally (e.g., intravenously, subcutaneously or intradermally).

For the treatment of medical conditions of the present invention described herein some embodiments the NK-1 receptor antagonist (e.g., orvepitant) is administered in a dose of about 0.5 to 60 mg per day. Preferably, it is 1 to 60 mg per day, more preferably 2.5 to 60 mg per day, more preferably 10 to 60 mg per day, more preferably 10 to 40 mg per day, more preferably 20 to 60 mg per day, more preferably 10 to 30 mg per day, more preferably 25 to 35 mg per day.

In certain embodiments, the NK-1 receptor antagonist (e.g., orvepitant) is administered in a dose of about 10 mg per day, about 15 mg per day, about 20 mg per day, about 25 mg per day, about 30 mg per day, about 35 mg per day, about 40 mg per day, about 45 mg per day, about 50 mg per day, about 55 mg per day, or about 60 mg per day.

It will be appreciated that it may be necessary to make routine variations to the dosage, depending on the age and condition of the patient and the precise dosage will be ultimately at the discretion of the attendant physician or veterinarian. The dosage will also depend on the route of administration.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

The pharmaceutical compositions of the present invention may be given in a single dose or multiple doses daily.

It is understood, however, that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time are well known in the art.

The optimal formulations can be determined by one skilled in the art depending upon considerations such as the particular ingredients and the desired dosage. See, for example, Remington's Pharmaceutical Sciences, 18th ed. (1990, Mack Publishing Co., Easton, Pa. 18042), pp. 1435-1712, and “Harry's Cosmeticology”, 8th ed. (2000, Chemical Publishing Co., Inc., New York, N.Y. 10016), the disclosure of each of which is hereby incorporated by reference herein in its entirety. Such formulations may influence the physical state, stability, rate of in vivo release, and rate of in vivo clearance.

In particular, the ability to formulate compositions capable of long term storage, without pre-mixing or compounding requirements prior to application, are also contemplated. Specifically, the present compositions remain unexpectedly stable in storage for periods including between about 3 months and about 3 years, about 3 months and about 2.5 years, between about 3 months and about 2 years, between about 3 months and about 20 months, and alternately any time period between about 6 months and about 18 months.

Thus, in another aspect, the invention provides a pharmaceutical composition comprising an NK-1 receptor antagonist and one or more pharmaceutically acceptable carriers or excipients for use in treating acute respiratory distress syndrome (ARDS), multiple organ dysfunction syndrome (MODS), sepsis, septic shock or systemic inflammatory response syndrome (SIRS).

Combination Therapies with an NK-1 Receptor Antagonist and Other Therapeutic Agents

It will be appreciated by those skilled in the art that the compound (I) or pharmaceutically acceptable salts thereof according to the invention may advantageously be used in combination with one or more other therapeutic agents, for instance:

For sepsis NK-1 receptor antagonists could be combined with anti-microbial therapy and all-purpose supporting treatment. Anti-microbial therapy includes anti-bacterials, anti-virals and anti-fungals depending on the source of the infection. All-purpose supporting treatment may include supplemental oxygen to prevent or treat hypoxia, invasive or non-invasive ventilation to treat respiratory failure, heparin to prevent deep-vein thrombosis, corticosteroids to reduce inflammation, anabolic therapies such as insulin and glutamine, renal replacement and electrolyte therapies, enteral or parenteral nutritional therapies, and if blood pressure is low indicative of septic shock then fluid volume therapies and vasopressor medications may be administered.

For SIRS supporting treatment may include supplemental oxygen to prevent or treat hypoxia, invasive or non-invasive ventilation to treat respiratory failure, heparin to prevent deep-vein thrombosis, corticosteroids to reduce inflammation, anabolic therapies such as insulin and glutamine, renal replacement and electrolyte therapies, enteral or parenteral nutritional therapies and if blood pressure is low then fluid volume therapies and vasopressor medications may be administered.

For ARDS, NK-1 receptor antagonists could be combined with supplemental oxygen, non-invasive ventilation or mechanical ventilation incorporating such measures as low tidal volumes, positive end expiratory pressure and a high inspiration to expiration time ratio, and extracorporeal membrane oxygenation (ECMO).

Furthermore NK-1 receptor antagonists for use in treatment of ARDS according to the invention may advantageously be used in combination with one or more other therapeutic agents, for example with corticosteroids, such as dexamethasone, methylprednisolone or hydrocortisone; with agents with anti-inflammatory/anti-fibrotic activity such as for example pirfenidone (5-methyl-1-phenylpyridin-2(1 H)-one) or with receptor kinase inhibitors such as nintedanib (methyl2-hydroxy-3-[N-[4-[methyl-[2-(4-methylpiperazin-1-yl)acetyl]amino]phenyl]-C-phenylcarbonimidoyl]-1H-indole-6-carboxylate).

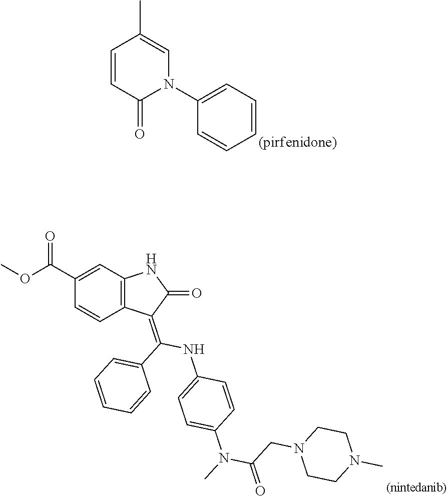

NK-1 receptor antagonists for use in treatment of ARDS according to the invention may advantageously be also used in combination with connective tissue growth factor (CTGF) inhibitors such as pamrevlumab; with synthetic analogues of pentraxin-2 (PTX2) (also called serum amyloid P component), that are modulators of fibrotic tissue, such as the recombinant form of the human PTX2 protein called PRM-151; with synthetic analogues of prostacyclin such as treprostinil; with anti-virals such as remdesivir; and immunomodulatory drugs (IMiDs) such as thalidomide.

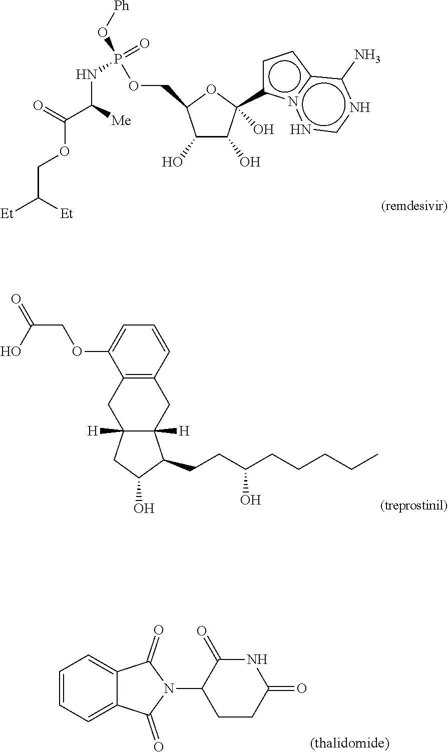

Thus, in some embodiments, the present invention provides a combination of an NK-1 receptor antagonist with one or more therapeutic agents and optionally with one or more pharmaceutically acceptable excipient(s) for use in treating a disease selecting from sepsis, septic shock, systemic inflammatory response syndrome (SIRS), acute respiratory distress syndrome (ARDS) or multiple organ dysfunction syndrome (MODS.

In some embodiments, the present invention provides a combination of an NK-1 receptor antagonist selected from orvepitant, aprepitant, fosaprepitant, rolapitant, netupitant, fosnetupitant, serlopitant, tradipitant or prodrug, metabolites or pharmaceutically acceptable salt thereof with one or more therapeutic agents and optionally with one or more pharmaceutically acceptable excipient(s) for use in treating a disease selecting from sepsis, septic shock, systemic inflammatory response syndrome (SIRS), acute respiratory distress syndrome (ARDS) or multiple organ dysfunction syndrome (MODS).

In some embodiments, the present invention provides a combination which comprises an NK-1 receptor antagonist with one or more therapeutic agents and optionally one or more pharmaceutically acceptable excipient(s) for use the treatment of ARDS.

In some embodiments, the present invention provides a combination of an NK1 receptor antagonist selected from orvepitant, aprepitant, fosaprepitant, rolapitant, netupitant, fosnetupitant, serlopitant, tradipitant or analogs, derivatives, prodrug, metabolites or pharmaceutically acceptable salt thereof with one or more therapeutic agents selected from corticosteroids, such as dexamethasone, methylprednisolone or hydrocortisone, with agents with anti-inflammatory/anti-fibrotic activity such as for example, pirfenidone, with receptor kinase inhibitors such as nintedanib, with connective tissue growth factor (CTGF) inhibitors such as pamrevlumab; with synthetic analogues of pentraxin-2 (PTX2) (also called serum amyloid P component), that are modulators of fibrotic tissue such as the recombinant form of the human PTX2 protein called PRM-151; with synthetic analogues of prostacyclin such as treprostinil; with anti-virals such as remdesivir and immunomodulatory drugs (IMiDs) such as thalidomide, and optionally one or more pharmaceutically acceptable excipient(s) for use in the treatment of ARDS.

In some embodiments the present invention provides a combination which comprises orvepitant, or pharmaceutically acceptable salt thereof and a second drug substance and optionally one or more pharmaceutically acceptable excipient(s) for the treatment of ARDS.

In some embodiments the present invention provides a combination which comprises orvepitant, or pharmaceutically acceptable salt thereof and one or more therapeutic agents selected from dexamethasone, methylprednisolone or hydrocortisone, pirfenidone, nintedanib, pamrevlumab, the recombinant form of the human PTX2 protein called PRM-151, treprostinil; remdesivir or thalidomide and optionally one or more pharmaceutically acceptable excipient(s) for use in the treatment of ARDS.

### EXAMPLES

Preclinical Studies

NK-1 Antagonists as a Method of Preventing and Treatment of ARDS

Orvepitant was evaluated in the established mouse model of ARDS; the LPS induced neutrophilia model of lung inflammation and injury.

The effects of vehicle control or orvepitant (250 mg/kg of orvepitant maleate anhydrous crystalline form [Form 1]), dosed orally 1 hour prior to challenge, on pulmonary inflammation induced by a single intranasal challenge dose (10 ug/kg) of either proinflammatory lipopolysaccharide (LPS), or phosphate-buffered saline (PBS) control, was investigated in female C57BL6J mice. See Table 1 for treatment groups. Animals were euthanised at either 4 or 24 hours, and inflammatory cell counts and mediators were assayed in bronchoalveolar lavage (BALF) from all animals.

TABLE 1
Overview of animal groups in LPS-induced neutrophilia
model study of orvepitant as a treatment for ARDS

| | | Treatment | Dose | | Euthanasia | |
|---|---|---|---|---|---|---|
| | | (po) (minus | (po, | Challenge | timepoint after | |
| Group | n | 1 hour) | mg/kg) | (in) (0 hr) | challenge (hour) | Readouts |
| 1 | 10 | Vehicle | — | PBS | 4 | Inflammatory |
| 2 | | Vehicle | — | LPS | | cell counts |
| 3 | | Orvepitant | 250 | LPS | | and mediators |
| 4 | | Vehicle | — | PBS | 24 | in BALF |
| 5 | | Vehicle | — | LPS | | |
| 6 | | Orvepitant | 250 | LPS | | |

Key: Bronchoalveolar lavage fluid (BALF); intranasal (in); lipopolysaccharide (LPS); number of female C57BL6J mice/group (n); per os (po); phosphate buffered saline (PBS).

Results

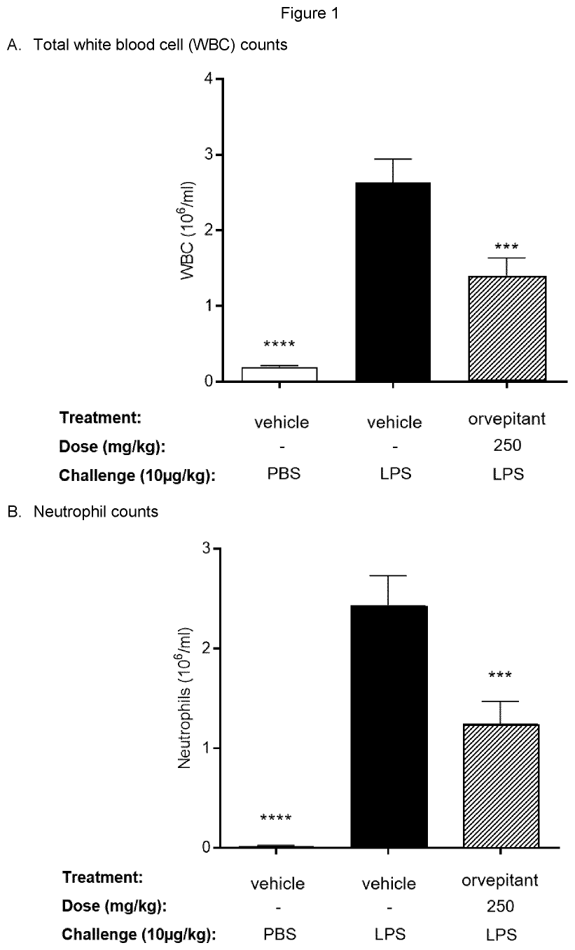

Total white blood cell and neutrophil counts together with a range of proinflammatory mediators in the bronchoalveolar lavage fluid (BALF) were significantly increased in LPS-challenged mice (Groups 2 & 5) compared to PBS-challenged animals (Group 1 & 4) at both 4 and 24 hours. Prior administration of orvepitant to mice in the LPS-challenged group significantly decreased both total white blood cell (WBC) (minus 50%; p<0.001) and neutrophil (minus 49%; p<0.001) counts at 24 hours (Group 3) compared to animals given vehicle control in the LPS-challenged (Group 5). See FIG. 1.

Consistent with this observation, orvepitant also reduced a range of proinflammatory mediators measured in the BALF at both 4 and 24 hours (Groups 3 & 6) compared to vehicle treated LPS-challenged animals (Groups 2 & 5). This data is summarised in Table 2.

TABLE 2
Summary data of effect of orvepitant (dose of 250 mg/kg administered
per os) compared to vehicle control group on inflammatory cell
counts and mediators in BALF at 4 and 24 hours following LPS
intranasal challenge. Statistical analysis was performed by
One Way ANOVA followed by Dunnetts's multiple comparison
of orvepitant/LPS group vs vehicle/LPS treated group. In some
analyses Grubbs' test was used to exclude outliers.

| | | 4 hr Statistical | | 24 hr Statistical | |
|---|---|---|---|---|---|
| | | significance (% | | significance (% | |
| | Assay | mean reduction) | | mean reduction) | |
| Cell counts | WBC | NS | | *** | (50%) |
| | Neutrophils | NS | | *** | (49%) |
| Mediators | IL-1β | NS | | p = 0.08 | (33%) |
| | IL-6 | ** | (44%) | p = 0.11 | (39%) |
| | IL17A | **** | (36%) | * | (47%) |

| | | | |
|---|---|---|---|
| TNFα | * | (26%) | NS |

| | | | |
|---|---|---|---|
| VEGF-A | NS | p = 0.09 | (32%) |
| MCP-1 | NS | ** | (56%) |
| TGF-β1 | NT | *** | (62%) |

Statistical significance:
* p < 0.05,
** p < 0.01,
*** p < 0.001
**** p < 0.0001.
Key: Bronchoalveolar lavage fluid (BALF); intranasal (in); lipopolysaccharide (LPS); not tested (NT) as analyte was below the limit of quantification; not significant (NS); number of female C57BL6J mice/group (n); per os (po); phosphate buffered saline (PBS).

Orvepitant ameliorated both inflammatory cell migration and reduced proinflammatory mediator production in the ARDS LPS induced neutrophilia model of lung inflammation and injury. As this model recapitulates inflammatory cascades that are associated with pulmonary inflammation and injury in ARDS then this data demonstrates that NK-1 antagonists may have the potential to be administered as a method of prevention and/or treatment of this acute pulmonary condition. Given LPS challenge by other routes of administration is also used to evaluate possible therapeutics for sepsis, septic shock, systemic inflammatory response syndrome (SIRS), and multiple organ dysfunction syndrome (MODS), the data presented also supports the potential utility of NK-1 antagonists in these conditions.

Clinical Studies

NK-1 Antagonists as a Method of Preventing and Treatment of ARDS

The efficacy of orvepitant as a method for treating ARDS is evaluated in a randomised double-blind study in which orvepitant is compared with standard of care. Patients hospitalised with severe acute dyspnoea of unknown cause or following an identifiable trigger event (eg acute infection, trauma pancreatitis) and objective evidence of worsening respiratory impairment (low arterial oxygen saturation on room air) are randomised to orvepitant or standard of care for 28 days. Efficacy is assessed using a composite endpoint of need for invasive ventilation or death, with efficacy established if there is a significant difference in favour of orvepitant in the number of subjects with one or other of the outcome criteria. Other endpoints assessed include serial arterial oxygen saturation measurements, time to invasive ventilation, duration of ICU admission and duration of invasive ventilation.

NK-1 Antagonists as a Method of Preventing and Treatment Septic Shock, MODS and SIRS

The efficacy of orvepitant as a method for treating septic shock, MODS and SIRS is evaluated in a randomised double-blind study in which orvepitant is compared with standard of care. Patients hospitalised with established or evolving septic shock, MODS or SIRS are randomised to orvepitant or standard of care for 28 days. Efficacy is assessed using a composite endpoint of all cause and 28 day ICU mortality, with efficacy established if there is a significant difference in favour of orvepitant in the number of subjects who survive for 28 days. Other endpoints assessed include organ dysfunction scores, duration of ICU admission and duration of invasive ventilation.

1. A method of treating a disease selected from sepsis, septic shock, systemic inflammatory response syndrome (SIRS), acute respiratory distress syndrome (ARDS) and multiple organ dysfunction syndrome (MODS) with an NK-1 receptor antagonist in patients in need thereof, said method comprising:
administering to said patients a therapeutically effective amount of said NK-1 receptor antagonist; and
treating said patients.

2. The method of claim 1, wherein the NK-1 receptor antagonist is selected from orvepitant, aprepitant, fosaprepitant, rolapitant, netupitant, fosnetupitant, serlopitant, tradipitant or prodrug, metabolites and pharmaceutically acceptable salt thereof.

3. The method of claim 1, wherein the disease is selected from sepsis, septic shock, acute respiratory distress syndrome (ARDS) and multiple organ dysfunction syndrome (MODS).

4. The method of claim 1, wherein the disease is acute respiratory distress syndrome (ARDS).

5. The method of claim 4, wherein the acute respiratory distress syndrome (ARDS) is due to or associated with a coronavirus infection.

6. The method of claim 5, wherein the coronavirus infection is the COVID-19 infection.

7. The method of claim 4, wherein the acute respiratory distress syndrome (ARDS) is due to or associated with acute exacerbations of interstitial lung diseases (AE-ILD).

8. The method of claim 7, wherein the acute exacerbations of interstitial lung diseases (AE-ILD) is acute exacerbations of idiopathic pulmonary fibrosis (AE-IPF).

9. The method of claim 2, wherein the NK-1 receptor antagonist is or comprises orvepitant or pharmaceutically acceptable salt thereof.

10. The method of claim 9, of wherein the NK-1 receptor antagonist is or comprises orvepitant maleate.

11. The method of claim 10, wherein the NK-1 receptor antagonist is or comprises orvepitant maleate crystalline form.

12. The method of claim 11, wherein the NK-1 receptor antagonist is or comprises orvepitant maleate anhydrous crystalline Form 1.

13. A method of treating a disease selected from sepsis, septic shock, systemic inflammatory response syndrome (SIRS), acute respiratory distress syndrome (ARDS) and multiple organ dysfunction syndrome (MODS) with a pharmaceutical composition comprising an NK-1 receptor antagonist and one or more pharmaceutically acceptable carriers or excipients in patients in need thereof, said method comprising:
administering to said patients a therapeutically effective amount of said pharmaceutical composition comprising NK-1 receptor antagonist and one or more pharmaceutically acceptable carriers or excipients; and
treating said patients.

14. The method of claim 1, wherein said NK-1 receptor antagonist is in combination with one or more therapeutic agents and optionally one or more pharmaceutically acceptable excipients.

15. The method of claim 14, wherein the NK-1 receptor antagonist is orvepitant or a pharmaceutical acceptable salt thereof, the disease is acute respiratory distress syndrome (ARDS) and the one or more therapeutic agents are selected from dexamethasone, methylprednisolone or hydrocortisone, pirfenidone, nintedanib, pamrevlumab; the recombinant form of the human PTX2 protein PRM-151, treprostinil, remdesivir and thalidomide.
